(19) United States
(12) Patent Application Publication
(10) Pub. No.: US 2006/0241492 A1
(43) Pub. Date: Oct. 26, 2006

(54) Method for medical imaging and a medical imaging system

(75) Inventors: Jan Boese, Eckental (DE); Martin Kleen, Furth (DE); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/374,831

(22) Filed: Mar. 14, 2006

(30) Foreign Application Priority Data
Mar. 18, 2005 (DE) 10 2005 012 699.5

(51) Int. Cl. A61B 6/00

(52) U.S. Cl. 600/473; 600/478

(57) ABSTRACT

To improve medical imaging with the aid of an intravascular catheter, an area to be examined is irradiated with the infrared light and an assigned scatter light signal is processed to form an image. To do this, OCT imaging with the aid of tissue-permeable light at a wavelength of approximately 1300 nm is combined with OCT imaging with the aid of blood-permeable light at a wavelength of approximately 1800 nm and/or with radio-optic imaging with the aid of an infrared camera (10B) with blood-permeable light at a wavelength of 1800 nm. This combined imaging catheter system opens up new and improved application possibilities in the medical field and the quality of the images received can be improved by the mutual correction of the particular image data.

### CROSS REFERENCE TO RELATED APPLICATIONS

This application claims priority to the German Application No. 10 2005 012 699.5, filed Mar. 18, 2005 which is incorporated by reference herein in its entirety.

### FIELD OF INVENTION

The invention relates to a method of medical imaging and a medical imaging system for recording intravascular images.

### BACKGROUND OF INVENTION

Images of the area of the vessels or organs of interest mainly using intravascular imaging systems are created for the medical treatment of vessels or organs of the human body. With this method, a catheter is normally introduced into the human body. An optical fiber cable is arranged within the catheter for optical imaging methods. The area to be examined is irradiated with infrared (IR) light. The light is reflected or scattered and applied to an evaluation unit as a light signal.

### SUMMARY OF INVENTION

A common method in this case is, for example, the optical coherence tomography (OCT) method. With this method of examination, the light particles scattered in the tissue are precisely filtered out using their interference capability. To do this, infrared light is irradiated vertical to the surface of the tissue in only a short coherence length, for example, of only an approximate 10 μm. The backscattered light is normally analyzed using an interferometric arrangement, for example, using a type of Michelson interferometer. Normally, light in the lightwave range of approximately 1300 nm is used for the OCT. By means of the OCT and the chosen lightwave range, tissue can be inspected down to a depth of a few millimeters. The OCT is therefore particularly suitable for a qualitative plaque assessment.

The problem with the chosen wavelength of 1300 nm is that blood is not permeable to IR light, because the light is scattered at a phase boundary between blood plasma and blood cells. When investigating blood vessels or organs filled with blood, such as the heart, the catheter must therefore have direct contact with the vessel wall of the organ or vessel under examination. Alternatively, there is also the possibility of keeping the blood away from the site under examination or replacing it by a liquid, such as sodium chloride solution, that is transparent with respect to the radiated light. The second possibility is normally used for vessel examinations.

A method is known from U.S. Pat. No. 6,178,346 B1 whereby a radio-optical image is taken with the aid of an infrared camera. In this case, a wavelength of approximately 1800 nm is used. Only a slight absorption and slight scatter of the light on the blood takes place in this wavelength range, so that the blood is transparent with respect to infrared light at this wavelength.

An object of the invention is to enable improved intravascular optical imaging.

The object is achieved by the claims. According to this, the intravascular images are obtained by irradiating the area to be examined with light in the infrared range using an optical fiber inside a catheter. The reflected or scattered light is applied as an assigned light signal via the optical fiber to an evaluation unit and is processed to generate images. The image information is evaluated, either alternately or simultaneously during an examination, using at least two of the following optional types of imaging.
Imaging with the aid of optical coherence tomography using light, the wavelength of which is chosen in such a way that the light is tissue-permeable. A wavelength in the area of approximately 1300 nm is particularly chosen for this.
Imaging with the aid of optical coherence tomography with light, the wavelength of which is chosen so that the light is blood-permeable. A wavelength in the 1800 nm range is appropriately chosen for this purpose.
Imaging whereby a radio-optical image is taken with the aid of an infrared camera (10B). In this case also blood-permeable light in a wavelength range of 1800 nm is preferably chosen.

A corresponding medical imaging system is in this case appropriately able to provide tissue-permeable and blood-permeable light for the examination. Furthermore, such a system is at the same time designed for performing the OCT and also for taking radio-optic images with the aid of an infrared camera.

By means of a combination of at least two of the imaging systems, mutually supplementary image information is obtained in a particularly advantageous manner, so that the medical personnel engaged in the examination are provided with more, and also more accurate, information on the vessel and/or organ to be examined, and within a uniform system.

In particular, the combined use of blood-permeable light and tissue-permeable light offers substantial advantages. For example, in just one examination it is possible to reliably, and comparatively quickly, search the tissue surfaces of vessels or organs for possible problem areas with the aid of the blood-permeable methods. If suspicious areas are detected, these can be examined more closely at the same time. In particular, the tissue can be examined in depth using the tissue-permeable OCT. Especially the combination of these imaging methods within an overall system, with only one catheter having to be introduced, enables a more precise and reliable result compared with the conventional simple examination methods.

In accordance with an appropriate development, an axial optical fiber is provided, by means of which the area to be examined is irradiated with light propagating mainly forward in the lengthwise direction of the catheter. By means of this axial optical fiber, it is therefore possible to examine tissue located in front of the catheter in the direction of feed. To be able to image the largest possible area of tissue, the axial optical fiber can, in accordance with an appropriate development, be rotated about the longitudinal axis of the catheter, so that a cone of irradiation with an approximate acceptance angle in the 60° range is generated.

As an alternative, or addition, to the essential longitudinal orientation of the catheter with only one axial optical fiber, an optical fiber bundle, or also an optical fiber array, aligned in the longitudinal direction of the catheter is provided, that irradiates a forward area of the tissue and thus light signals can be recorded and evaluated from this.

Preferably, particularly in addition to the axial irradiation of the tissue to be examined with the axial optical fiber or with the optical fiber bundle, a radial irradiation is provided. For this purpose the optical fiber cable includes a radial optical fiber that has a light exit aperture directed radially relative to the longitudinal direction of the catheter. In this case, the radial optical fiber is appropriately rotatable about the longitudinal axis of the catheter. In particular, the combination of axial light propagation and radial light propagation enables concealed structures to be examined and detected, that are not detected simply by an axial “direction of view”. Because they are located, for example, in the shaded area behind obstacles, the concealed structures cannot be detected by axially emerging light. Further advancement of the catheter with a succeeding radial irradiation is required to obtain an image of the concealed structures.

To facilitate the image evaluation by the medical personnel, in an appropriate development the various pieces of image information are compared and processed to form a common image if required. Particularly where both blood-permeable light and tissue-permeable light are used, supplementary, complimentary pieces of information are obtained that are combined in an image to improve the image quality. For this combined visualization, the image data obtained by various types of imaging is suitably merged. Appropriate known methods are used for this purpose, such as are used for image evaluating systems in medicine.

In a preferred embodiment, the catheter is moved during the examination within the vessel or organ so that it takes up different positions. Image information is acquired at the different positions of the catheter, from which a three-dimensional image data record is generated. Three-dimensional images of the anatomy of a vessel or organ that are reliable and easy to evaluate are obtained in this way.

Appropriately, the position of the catheter is determined with the aid of a location sensor for a precise determination of the actual position of the catheter and thus for creation of the most reliable 3D data record. A location sensor of this kind is, for example, mounted directly on the point of the catheter and transmits electromagnetic location signals that are received and evaluated by an appropriate receiver.

To obtain additional information on the tissue to be examined, a preferred development is provided that, in addition to the optical imaging method, also uses an intravascular ultrasound method of imaging (IVUS).

Preferably, the image data obtained using the intravascular catheter system is additionally compared with the image data from other imaging non-intravascular systems and combined as required. Such further imaging systems are, for example, computer tomography, magnetic resonance examination, 3D or 2D angiography or the extravascular ultrasound examination. By means of a combination with these other imaging systems, information that is therefore reliable and comprehensive is obtained regarding the vessels or organs examined.

The object is further achieved in accordance with the invention by a medical imaging system for taking intravascular images. Preferred embodiments are given in the dependent claims. The advantages listed with regard to the method and the preferred embodiments can also be transferred appropriately to the medical imaging system.

### BRIEF DESCRIPTION OF THE DRAWINGS

Exemplary embodiments of the invention are explained in more detail in the following with the aid of drawings. These schematic illustrations are as follows:

FIG. 1 A possible layout of a medical imaging system for obtaining intravascular images,

FIG. 2 A highly schematized representation of a catheter head,

FIG. 3 An illustration showing the principle of OCT imaging using a fiber bundle,

FIG. 4 A schematic representation of OCT imaging using an axial optical fiber,

FIG. 5 A schematic representation of OCT imaging using a radial optical fiber,

FIG. 6 A schematic representation of optical imaging using a fiber bundle.

### DETAILED DESCRIPTION OF INVENTION

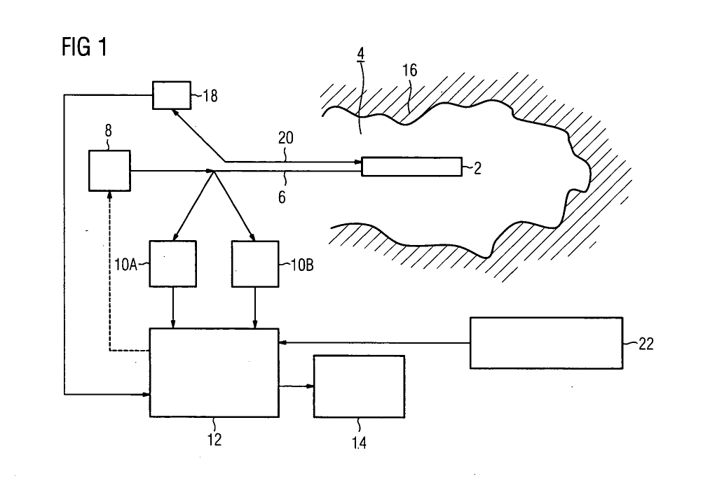

A medical imaging system shown in FIG. 1 has a catheter 2 that during the examination is inserted into the vessel to be examined 4 of a human body. The catheter 2 is connected via an optical fiber cable 6 to a supply unit 8. Infrared light is supplied to the optical fiber cable 6 via this supply unit. The supply unit 8 is designed so that it can supply infrared light both in the 1300 nm wavelength range and also approximately in the 1800 nm wavelength range.

The imaging system also includes a first reception and evaluation unit 10A, that is designed for imaging using the optical coherence tomography (OCT) imaging method. Furthermore, the system has a second reception and evaluation unit 10B, that is designed as an infrared camera for radio-optic imaging. The evaluation units process received light signals to obtain image information that is transmitted to a central computer unit 12. In the computer unit 12, this image information is further processed and displayed for visualization on a display element 14, especially a screen. The individual component can, if appropriate, be integrated into a common unit.

The system described so far, with the catheter 2, the supply unit 8, the evaluation unit 10A, the IR camera 10B, the computer unit 12 and the display element 14 form a medical imaging system for obtaining optical images using different types of imaging. The system enables different wavelengths to be used for the IR light and preferably at the same time combines OCT imaging with radio-optic imaging.

Of particular advantage is the possibility of supplying light both with a wavelength of 1300 nm and a wavelength of 1800 nm. In this way, the vessel 4 to be examined can be irradiated with light that in the first case (1300 nm) is suitable for penetrating into the tissue 16 of the vessel 4 (tissue-permeable light). In the second case (1800 nm), the light is able to penetrate the blood (blood-permeable light).

These different properties of the light, i.e. the tissue-permeable (but not blood-permeable) and the blood-permeable (but not tissue-permeable) are due to the wavelength-specific scatter behavior of the blood or tissue. The combination of imaging using blood-permeable light and tissue-permeable light enables supplementary and complimentary image information to be obtained that enables the particular condition of the vessel 4 to be more reliably assessed.

In addition to the optical intravascular imaging system already described, in the exemplary embodiment this is additionally combined with an imaging intravascular ultrasound system (IVUS). For this purpose, an IVUS unit 18 is provided that controls the generation and evaluation of the reflective ultrasonic waves. For the intravascular ultrasonic imaging, an ultrasonic transducer is normally fitted to the point of the catheter, through which ultrasound is applied to the structure to be examined. At the same time, the reflected sound signal from the transducer is converted to an electrical signal and passed via the cable 20 to the IVUS unit 18 for evaluation. Where space and conditions permit, the ultrasonic transducer is preferably arranged in the point of the catheter together with optical components for the optical imaging. As an alternative, it is also possible to withdraw the components for optical imaging from the catheter 2 inserted into the vessel 4 and, in their place, insert the ultrasonic components into the catheter 2.

The ultrasonic signals are prepared by the IVUS unit 18 as image information and transmitted to the computer unit 12, where they are further processed.

Finally, it is also possible for the computer unit 12 to receive further image information that was obtained by a non-intravascular imaging system 22, such as computer tomography, magnetic resonance, 2D, 3D angiography, etc.

For a comprehensive image evaluation, all the image information supplied to the computer unit 12 is processed using known image processing methods and combined to form a joint picture as required using the methods of image fusion.

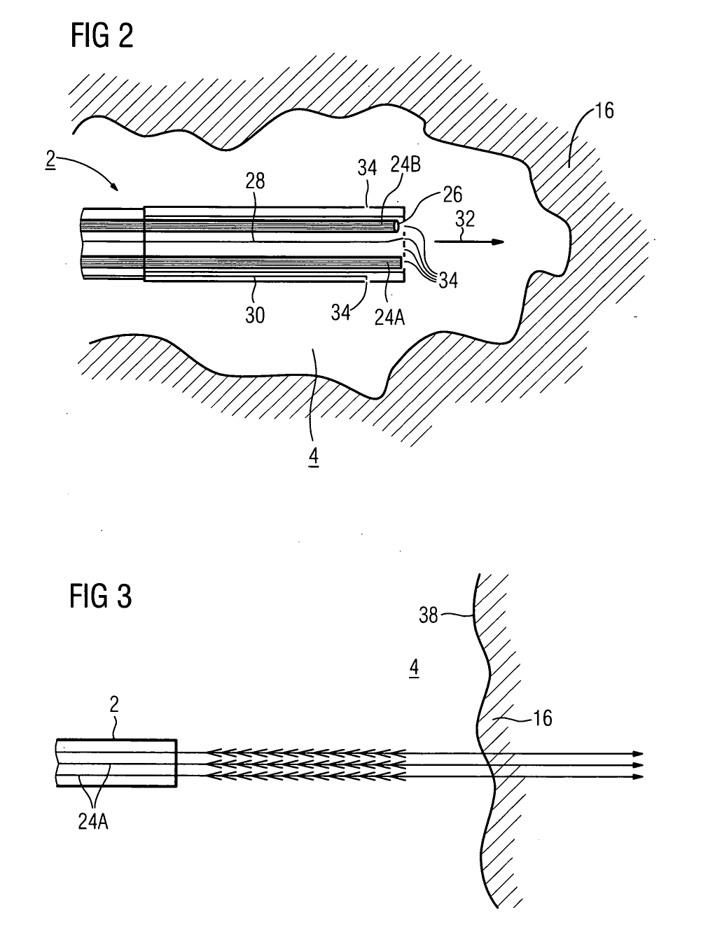

FIG. 2 shows a roughly simplified and schematic representation of a catheter point in which several optical components are integrated. These are a first fiber bundle 24A for the OCT imaging, a second fiber bundle 24B with an optical lens 26 at the end for radio-optic imaging, an axial fiber 28 and a radial optical fiber 30. Both fiber bundles 24A,B each have several optical fibers and are aligned in the longitudinal direction 32 on the catheter 2. The axial optical fiber 28 is also essentially oriented in the longitudinal direction 32. The axial optical fiber 28 can also be rotated about the longitudinal axis of the catheter 2, so that a radiation area that is approximately cone-shaped can be generated with this axial optical fiber 28. Both fiber bundles 24A,B and the axial optical fiber 28 radiate the infrared light in the longitudinal direction 32 forward through the front of the catheter 2. Light apertures 34 are provided for this purpose. As an alternative to discrete light apertures, the catheter wall can consist completely of a material that is transparent for IR light.

In contrast to this, the direction of irradiation defined by the radial fiber 30 is oriented vertical to the longitudinal direction 32. The radial optical fiber 30 can also be rotated about the longitudinal axis 32 of the catheter 2 and radiates the IR light radially relative to the longitudinal direction 32 via a circular light aperture 34.

In the exemplary embodiment in FIG. 2, the different optical components, i.e. both fiber bundles 24A,B and both optical fibers 28,30 are jointly integrated in a catheter 2. As an alternative to this, it is also possible to combine only the required subcombination of the optical components together in a catheter 2, which reduces the overall space requirement.

In principle, it is possible to provide both types of light, i.e. blood-permeable light and the tissue-permeable light through the same fiber bundle 24B or the same optical fibers 28, 30. As an alternative, it is also possible to use separate optical fibers for each of the two types of light. The irradiation of the tissue 16 with blood-permeable light on the one hand and tissue-permeable light on the other takes place, for example, alternately or simultaneously. In the case of simultaneous irradiation using different wavelengths via the same optical fibers, the different signals are separated using suitable frequency filters or other filters for the evaluation.

With the aid of FIG. 3 to 6, the different types of optical irradiation are explained in turn in the following.

In accordance with the variant shown in FIG. 3, the tissue 16 is irradiated, for OCT imaging, via the first fiber bundle 24A with tissue-permeable light at a wavelength of approximately 1300 nm. This fiber bundle 24A with several optical fibers thus represents an OCT array. Because of the chosen light wavelength of 1300 nm, the light penetrates into the tissue and there it is scattered. The scattered light is collected by the fiber bundle 24B as a scattered light signal and transmitted to the evaluation unit 10A. If a blood-filled vessel 4 is being examined, the intermediate space between the catheter 2 and the tissue 16 is initially full of blood. For the OCT examination using light that is tissue-permeable but blood impermeable, it is necessary for the intermediate space 36 to be flushed, i.e. the blood is replaced by a flushing liquid.

With the arrangement described in FIG. 3, it is also possible to irradiate the tissue 16 with blood-permeable light with a wavelength of approximately 1800 nm. In this case, the light is scattered or reflected at the interface 38 to the tissue 16 and the corresponding light signal is again sent back to the evaluation unit 10A.

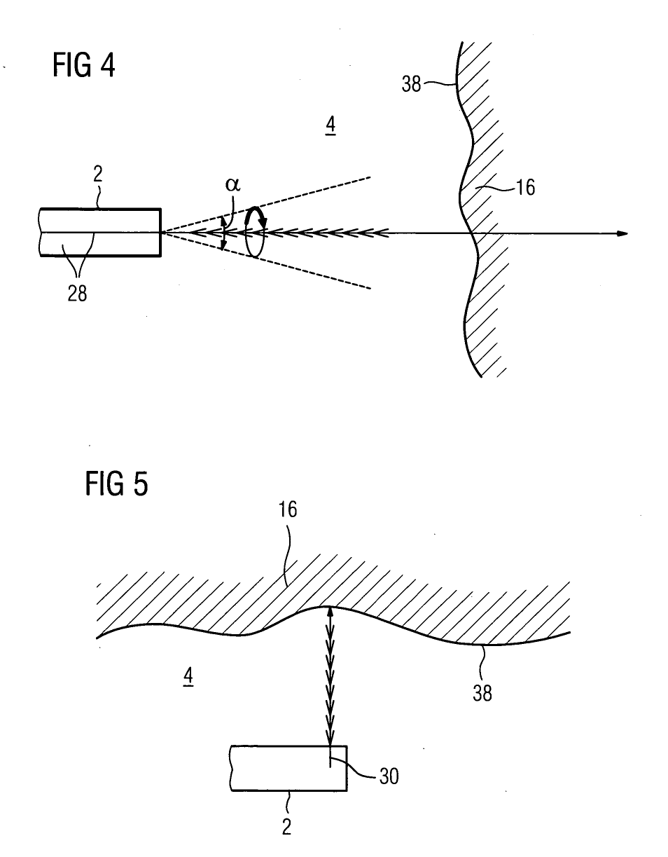

With the exemplary embodiment shown in FIG. 4, the tissue 16 is again irradiated with tissue-permeable light through the axial optical fiber 28. The axial optical fiber 28 is rotated about the longitudinal axis of the catheter to generate a light cone 40, to depict a flat image section. The light cone, for example, has an acceptance angle α of about 60°. In this case also, it is possible to use the tissue-permeable light and the blood-permeable light either at the same time or alternately.

In the exemplary embodiment in FIG. 5, the tissue 16 is irradiated with blood-permeable light through the radial optical fiber 30, for OCT imaging. The blood-permeable light is scattered or reflected at the interface 38. It is also possible in this case to irradiate the tissue 16 with tissue-permeable light either alternately or in parallel.

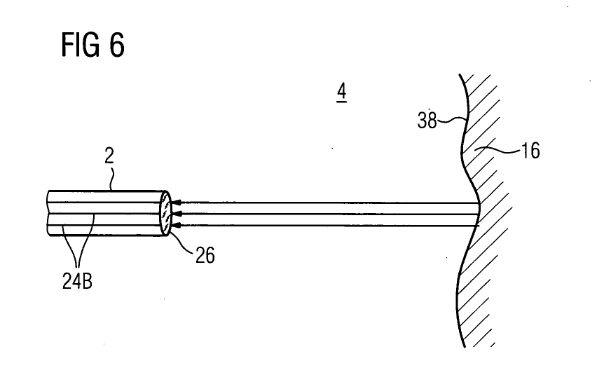

With the variant embodiment shown in FIG. 6, the tissue 16 is irradiated with blood-permeable light through the second fiber bundle 24B for optical imaging. The light signals reflected at the interface 38 are collected via the lens 26 or by an objective, injected into the second fiber bundle 24B and transmitted to the infrared camera 10B. The use of the tissue-permeable light is less useful in this case because no evaluatable image information is obtained due to the scatter effect in the tissue when purely radio-optical means are used. In principle, it is possible to also perform the optical imaging with the individual optical fibers 28, 30, but to obtain a good image quality an optical system consisting of the second fiber bundle 24B and lens 26 is advantageous.

In total there are therefore several possible combinations (variants) of the different kinds of examination, as can be seen in the following table 1, and in fact the tissue-permeable OCT imaging (i) can be carried out with the fiber bundle 24A (variant A), with the axial fiber 28 (variant D) and with the radial fiber 30 (variant F). In a similar manner, it is also possible to carry out blood-permeable OCT imaging (ii) using the three fiber variants (variants B, E, G). Finally, radio-optic, blood-permeable imaging (iii) is possible using the fiber bundle 24B (variant C).

TABLE 1

| | OCT | OCT | Optical |
|---|---|---|---|
| | tissue-permeable | blood-permeable | blood-permeable |
| | (i) | (i) | (ii) |
| Fiber bundle 24 | A | B | C |
| Axial fiber 28 | D | E | — |
| Radial fiber 30 | F | G | — |

To obtain better image information compared with the conventional intravascular optical imaging methods, at least two of the three types of imaging, OCT tissue-permeable (i), OCT blood-permeable (ii), optical blood-permeable (iii) are combined. Because of the different types of irradiation using the fiber bundle 24A, B or the single fibers 28, 30, a variety of possible combinations are available that in each case can be used in almost any combination to suit the special application and required result.

The following Table 2 is an overview of significant combinations of two from the individual variation possibilities of variants A-G arising from Table 2. In this case, Table 2 is to be read in such a way that the cells marked X represent combinations of two of variants of the particular line with variants of the particular column. The cells marked with a dot are merely mirror images of the cells marked with X.

TABLE 2

| | A | B | C | D | E | F | G |
|---|---|---|---|---|---|---|---|
| A | — | X | X | | X | X | |
| B | • | — | | X | | X | X |
| C | • | | — | X | | X | X |
| D | | • | • | — | X | X | X |
| E | • | | | • | — | X | X |
| F | • | • | • | • | • | — | X |
| G | | • | • | • | • | • | — |

The particular advantages or applications of some of the selected combinations of two are shown in the following:

AC

This combination is used particularly for visualizing an inner wall of blood-filled cavernous organs, such as the endocardium of the heart. In this case, the catheter of the optical blood-permeable infrared imaging (iii) is inserted into the cavernous organ, e.g. the ventricle of the heart. When a lesion is detected, the catheter 2 is moved so close to the inner wall of the cavernous organ (endocardium of the ventricle of the heart) forming the interface 38 that the catheter 2 contacts the inner wall. The structure of the tissue of the inner wall, for example lesions caused by ablation, is then depicted using tissue-permeable OCT imaging by means of the fiber bundle 24A.

BD

This combination enables a perspective record of the lumen of the vessel 4 while at the same time enabling a radial examination of the radial vessel wall for plaque formation. By means of this combination, a reliable 3D representation of the vessel 4 to be examined is obtained by moving the catheter 2 within the vessel 4, particularly when withdrawing the catheter 2 from the vessel 4. For this purpose, the different pieces of image information are suitably combined and processed.

BG

With this combination of two blood-permeable OCT imaging variants, a three-dimensional image, as with the previously explained combination, is enabled when advancing or withdrawing the catheter. By combining the radial examination with a forward-directed axial examination, structures hidden behind edges or waves are also covered by the axial examination.

BF

With this combination, the OCT fiber bundle 24A takes pictures of the lumen of the vessel 4 using blood-permeable light, with qualitative pictures of the tissue 16 or plaque being generated at the same time as required, by means of the rotating radial OCT fiber 30.

CD

The application of this combination corresponds approximately to the AC combination described above.

CG

This combination can be compared with the BG combination with regard to its application, because in this case also the image of the vessel 4 or of the blood-filled cavernous organ is taken using optical blood-permeable infrared imaging. Radial images around the catheter 2 are generated at the same time using the blood-permeable OCT and the radial fiber 30. In this way, sections of the vessel are also detected that could not be detected using optical imaging with a forward direction of view.

CF

With this combination, an image of the lumen of the vessel 4 is obtained using optical blood-permeable imaging. At the same time, or if required, a plaque analysis is carried out using the radial tissue-permeable OCT. This combination is therefore comparable with the BF combination with regard to this application.

DE

In this case, a tissue-permeable axial fiber 28 is combined with a blood-permeable axial fiber 28. Two different optical fibers can be provided for this, of which one, or both, can rotate about the longitudinal axis in order to depict the largest possible area. In principle, it is also possible to radiate both different kinds of light through one and the same fiber. This combination, for example, provides the possibility of imaging the endocardium during a single movement of the catheter 2 within a ventricle of the heart using blood-permeable OCT imaging. Immediately the catheter 2 has wall contact, i.e. touches the interface 38, a switch to tissue-permeable light takes place in order to obtain an image of tissue lesions.

EG

This combination corresponds essentially to the BG combination with regard to its application.

EF

This combination corresponds essentially to the BF combination with regard to its application.

GF

With this combination, the radial tissue 16 is examined using tissue-permeable and blood-permeable light, alternately or simultaneously, via the radial optical fiber 30 or, as necessary, using two separate radial optical fibers 30. Because complimentary image information is obtained in this way, the images generated with the aid of the blood-permeable OCT imaging are appropriately corrected using the images of the tissue-permeable OCT imaging or vice versa. This combination is useful for investigating blood-filled vessels, with the blood-permeable OCT imaging used to take an image of the lumen of the vessel and the OCT imaging using tissue-permeable light being used at the same time to carry out a plaque examination.

Particularly in combinations of variants where a variant with blood-permeable light is combined with a variant with tissue-permeable light, a targeted and steady examination of a vessel 4, for example with regard to deposits on the vessel wall, can be carried out. To do this, the catheter is first inserted through the blood-filled vessel 4 with the aid of the OCT or optical imaging with blood-permeable light. Immediately suspicious areas are detected, either the catheter 2 is moved to the wall or a changeover to tissue-permeable OCT imaging takes place. Alternatively, when a suspicious area is found, the intermediate space 36 is flushed with a flushing liquid, particularly a sodium chloride solution.

The system described here combines the advantages of OCT imaging with blood-permeable light with those of the OCT imaging with tissue-permeable light and also the advantages of the optical infrared imaging with blood-permeable light in a combined imaging catheter system. This particularly supports applications in vessels or blood-filled organs (e.g. heart), whereby on the one hand lesions or deposits are qualitatively imaged by the tissue-permeable OCT imaging, and on the other hand the surface/interface 38 of organs is imaged with the aid of the OCT or radio-optic imaging using blood-permeable light.

In addition to the new application possibilities, the described combined catheter system also offers the possibility of improving the quality of the received images by mutual correction of the image data received from the different variants. Finally, the image data received from the optical imaging catheter system is improved and corrected using image data from other imaging systems, such as IVUS or non-intravascular systems 22.

1.-15. (canceled)

16. A method of acquiring medical images, comprising:
inserting a catheter into a vessel, the catheter having an optical fiber;
irradiating an examination area with infrared light using the optical fiber;
acquiring a response light signal from the examination area;
transmitting the response light signal to an evaluation unit; and
processing the transmitted response light signal by the evaluation unit to generate a medical image, wherein during a medical examination at least two imaging procedures are executed alternately or in parallel, the at least two imaging procedures selected from the group consisting of irradiating the examination area using optical coherence tomography including infrared light having a wavelength such that the infrared light permeates tissue, irradiating the examination area using optical coherence tomography including infrared light having a wavelength such that the infrared light permeates blood and acquiring the response light by an infrared camera for generating a radio-optic image.

17. The method in accordance with claim 16, wherein a wavelength of the infrared light is 1300 nm such that the infrared light permeates the tissue.

18. The method in accordance with claim 16, wherein a wavelength of the infrared light is 1800 nm such that the infrared light permeates blood.

19. The method in accordance with claim 16, wherein the optical fiber has an axial optical fiber for irradiating the examination area with the infrared light propagating essentially in a longitudinal direction relative to the catheter.

20. The method in accordance with claim 19, wherein the axial optical fiber is rotated about a longitudinal axis of the catheter.

21. The method in accordance with claim 16, wherein the optical fiber is an optical fiber bundle including a plurality of individual optical fibers having light exit apertures, the light exit apertures oriented in a longitudinal direction of the catheter.

22. The method in accordance with claim 16, wherein the optical fiber has a radial optical fiber for irradiating the examination area with the infrared light propagating essentially radially relative to a longitudinal direction of the catheter.

23. The method in accordance with claim 22, wherein the radial optical fiber is rotated about a longitudinal axis of the catheter.

24. The method in accordance with claim 16, wherein a merged image is generated based on the at least two imaging procedures.

25. The method in accordance with claim 16, further comprising:
moving the catheter during the medical examination; and
generating a three-dimensional image data record based on imaging data obtained at a plurality of positions of the catheter.

26. The method in accordance with claim 25, further comprising determining a current position of the catheter using a location sensor.

27. The method in accordance with claim 16, further comprising executing an intravascular ultrasonic examination using the catheter.

28. The method in accordance with claim 16, further comprising:
executing at least one further imaging procedure during the medical examination; and
generating a merged imaged based on the at least two imaging procedures and the further imaging procedure.

29. A medical imaging system for acquiring intravascular medical images, comprising:
a catheter having an optical fiber;
a light supply unit connected to the optical fiber for supplying infrared light to the optical fiber; and
an evaluation unit connected to the optical fiber for evaluating a response light signal, wherein the light supply unit is configured to simultaneously or alternately supply the infrared light at at least a first and a second wavelength, the first wavelength chosen such that the infrared light permeates blood and the second wavelength chosen such that the infrared light permeates tissue.

30. The medical imaging system in accordance with claim 29, wherein the system is configured for employing optical coherence tomography for acquiring the intravascular medical images using the infrared light having the first and second wavelengths and using a radio-optical image acquired by an infrared camera.

31. The medical imaging system in accordance with claim 29, wherein the system is configured for intravascular ultrasonic imaging.
